(19) United States
(12) Patent Application Publication
(10) Pub. No.: US 2015/0152441 A1
(43) Pub. Date: Jun. 4, 2015

(54) METHODS AND SYSTEMS FOR IMPROVING FERMENTATION EFFICIENCY

(71) Applicant: LanzaTech New Zealand Limited, Skokie, IL (US)

(72) Inventors: Michael Anthony Schultz, Skokie, IL (US); Paul Alvin Sechrist, Skokie, IL (US); Bjorn Daniel Heijstra, Skokie, IL (US)

(21) Appl. No.: 14/556,132

(22) Filed: Nov. 29, 2014

Related U.S. Application Data
(60) Provisional application No. 61/910,143, filed on Nov. 29, 2013.

(51) Int. Cl. C12P 7/08; C12P 7/54; C12P 7/18

(52) U.S. Cl. CPC C12P 7/08; C12P 7/18; C12P 7/54

(57) ABSTRACT

Processes, as well as associated systems, are disclosed for biological conversion of CO into desired end products such as ethanol and 2,3-butanediol. The pre-treatment of industrial gas streams comprising CO, such that the level of contaminants selected from the group comprising benzene, toluene, xylene and ethylbenzene, are maintained below a predetermined liquid level in the fermentation broth, has been shown to have positive effects on the fermentation process.

### FIELD OF THE INVENTION

This invention relates to systems and methods for improving efficiency in processes including microbial fermentation. In particular, the invention relates to improving efficiency in processes including microbial fermentation of a gaseous substrate comprising CO by providing the substrate such that the level of BTEX constituents are maintained below a predetermined liquid level in a fermentation broth.

### BACKGROUND OF THE INVENTION

Ethanol is rapidly becoming a major hydrogen-rich liquid transport fuel around the world. Worldwide consumption of ethanol in 2005 was an estimated 12.2 billion gallons. The global market for the fuel ethanol industry has also been predicted to grow sharply in future, due to an increased interest in ethanol in Europe, Japan, the USA, and several developing nations.

For example, in the USA, ethanol is used to produce E10, a 10% mixture of ethanol in gasoline. In E10 blends, the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, and as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, while the cultivation of starch or sucrose-producing crops for ethanol production is not economically sustainable in all geographies. Therefore, it is of interest to develop technologies to convert lower cost and/or more abundant carbon resources into fuel ethanol.

CO is a major, low cost, energy-rich by-product of the incomplete combustion of organic materials such as coal or oil and oil derived products. For example, the steel industry in Australia is reported to produce and release into the atmosphere over 500,000 tonnes of CO annually. Additionally or alternatively, CO rich gas streams (syngas) can be produced by gasification of carbonaceous materials, such as coal, petroleum and biomass. Carbonaceous materials can be converted into gas products including CO, CO₂, H₂ and lesser amounts of CH₄ by gasification using a variety of methods, including pyrolysis, tar cracking and char gasification. Syngas can also be produced in a steam reformation process, such as the steam reformation of methane or natural gas.

Catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source, all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce ethanol from gases (Abrini et al., Archives of Microbiology 161, pp 345-351 (1994)).

However, ethanol production by micro-organisms by fermentation of gases is typically associated with co-production of acetate and/or acetic acid. As some of the available carbon is typically converted into acetate/acetic acid rather than ethanol, the efficiency of production of ethanol using such fermentation processes may be less than desirable. Also, unless the acetate/acetic acid by-product can be used for some other purpose, it may pose a waste disposal problem. Acetate/acetic acid is converted to methane by micro-organisms and therefore has the potential to contribute to GHG emissions.

WO2007/117157, WO2008/115080 and WO/2009/058028, the disclosure of which are incorporated herein by reference, describe processes that produce alcohols, particularly ethanol, by anaerobic fermentation of gases containing carbon monoxide. Acetate produced as a by-product of the fermentation process described in WO2007/117157 is converted into hydrogen gas and carbon dioxide gas, either or both of which may be used in the anaerobic fermentation process. WO/2009/058028 discloses the use of untreated industrial gas streams as the carbon monoxide source for the fermentation process.

The fermentation of gaseous substrates comprising CO, to produce products such as acids and alcohols, typically favours acid production. Alcohol productivity can be enhanced by methods known in the art, such as methods described in WO2007/117157, WO2008/115080, WO2009/022925 and WO2009/064200, which are fully incorporated herein by reference.

U.S. Pat. No. 7,078,201 and WO 02/08438 also describe improving fermentation processes for producing ethanol by varying conditions (e.g. pH and redox potential) of the liquid nutrient medium in which the fermentation is performed. As disclosed in those publications, similar processes may be used to produce other alcohols, such as butanol.

Microbial fermentation of CO in the presence of H₂ can lead to substantially complete carbon transfer into an alcohol. However, in the absence of sufficient H₂, some of the CO is converted into alcohol, while a significant portion is converted to CO₂ as shown in the following equations:

6CO+3H₂O→C₂H₅OH+4CO₂

12H₂+4CO₂→2C₂H₅OH+6H₂O

The production of CO₂ represents inefficiency in overall carbon capture and if released, also has the potential to contribute to Green House Gas emissions. Furthermore, carbon dioxide and other carbon containing compounds, such as methane, produced during a gasification process may also be released into the atmosphere if they are not consumed in an integrated fermentation reaction.

It is an object of the present invention to provide system(s) and/or method(s) that overcomes disadvantages known in the art and provides the public with new methods for the optimal production of a variety of useful products.

### SUMMARY OF THE INVENTION

One aspect of the invention provides a system and method for the pre-treatment of an industrial waste gas stream, the method comprising;
a) flowing an industrial waste gas stream comprising CO, naphthalene and at least one contaminants selected from the group comprising benzene, toluene, xylene and ethyl benzene (BTEX) to an activated carbon bed unit, wherein at least a portion of the naphthalene is removed from the gas stream;
b) passing the naphthalene depleted gas stream of (a) to an adsorption unit, wherein at least a portion of the at least one BTEX contaminants is removed from the gas stream to provide a BTEX contaminant depleted reactor stream;
c) passing the reactor stream to a bioreactor containing a fermentation broth comprising a culture of at least one microorganism; and
d) fermenting the BTEX depleted reactor stream to produce one or more products.

In one embodiment the one or more products is selected from the group of ethanol, acetic acid (acetate), 2,3-butanediol, butanol, isopropanol, isoprene, lactate, succinate, methyl ethyl ketone (MEK), propanediol, 2-propanol, acetoin, isobutanol, citramalate, butadiene, poly lactic acid, isobutylene, 3-hydroxy propionate (3HP), acetone, and fatty acids. In a preferred embodiment, the culture produces one or more of ethanol, acetate, and 2,3-butanediol.

In one embodiment of the reaction, the activated carbon bed uses a carbon dioxide gas as the regeneration gas. In one embodiment of the invention the activated carbon bed unit comprises at least two carbon beds. In one embodiment of the invention, the activated carbon beds are cycled such that one carbon bed is in use, whilst a second carbon bed is being regenerated.

In one embodiment of the reaction, the adsorption unit is a Pressure Swing Adsorption (PSA) unit. In one embodiment, the adsorption unit combines Pressure Swing Adsorption and Temperature Swing Adsorption. In certain embodiments, the cycle time in the adsorption unit is increased.

In one embodiment of the reaction, at least a portion of CO₂ gas contained in the industrial waste gas stream is removed by the adsorption unit to provide a CO enriched reactor stream. In one embodiment at least a portion of the CO₂ removed by the adsorption unit is passed back to the activated carbon bed for use a regeneration gas.

In one embodiment, the amount of BTEX in the reactor stream is limited, such that the total BTEX contaminant concentration in the fermentation broth is less than 100 ppm, or less than 80 ppm, or less than 60 ppm, or less than 40 ppm, or less than 20 ppm.

In one embodiment of the invention there is substantially no BTEX contaminants in the reactor stream passed to the bioreactor. The term “substantially no” is intended to encompass gas streams containing trace levels of BTEX contaminants. For example, the stream may comprise less than 5 ppm.

In one embodiment, at least a portion of the at least one contaminant in the BTEX stream is recovered. In one embodiment, the recovered BTEX component is selected from benezene, toluene and xylene. In one embodiment at least a portion of Benzene in the BTEX stream is recovered.

In one embodiment, the fermentation reaction produces an exit stream comprising CO₂ and/or H₂. In one embodiment at least a portion of the CO₂ is passed to the activated carbon bed unit for use as a regeneration gas.

In one embodiment of the invention, the industrial waste gas is selected from the group consisting of ferrous metal product manufacturing waste gas, biomass synthesis gas, coke oven gas, coal syngas, municipal solid waste syngas and COREX gas. COREX gas is the gas by product produced by the smelting of iron ore and/or non-coking coal by the Corex smelting process.

In one embodiment of the invention the industrial waste gas is a steel manufacturing process waste gas.

### BRIEF DESCRIPTION OF THE DRAWINGS

FIG. 1. shows a modular diagram of the method and system in accordance with one embodiment of the invention;

FIG. 2. shows toluene accumulation levels in a fermentation broth in accordance with Example 1;

FIG. 3 shows the effect of toluene accumulation on CO uptake in accordance with Example 1;

FIG. 4 shows the effect of toluene accumulation on the pH of the fermentation in accordance with Example 1;

FIG. 5 shows benzene accumulation levels in a fermentation broth in accordance with Example 1;

FIG. 6 shows gas the effect of benzene accumulation on CO uptake in accordance with Example 1;

FIG. 7 shows the effect of benzene accumulation on pH of the fermentation in accordance with Example 1.

### DETAILED DESCRIPTION OF THE INVENTION

### Definitions

Unless otherwise defined, the following terms as used throughout this specification are defined as follows:

The term “carbon capture” as used herein refers to the sequestration of carbon compounds including CO₂ and/or CO from a stream comprising CO₂ and/or CO and either:

converting the CO₂ and/or CO into products; or

converting the CO₂ and/or CO into substances suitable for long term storage; or

trapping the CO₂ and/or CO in substances suitable for long term storage;

or a combination of these processes.

The term “substrate comprising carbon monoxide” and like terms should be understood to include any substrate in which carbon monoxide is available to one or more strains of bacteria for growth and/or fermentation, for example.

“Gaseous substrates comprising carbon monoxide” include any gas which contains carbon monoxide. The gaseous substrate will typically contain a significant proportion of CO, preferably at least about 5% to about 100% CO by volume.

The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Bubble Column, Gas Lift Fermenter, Static Mixer, a circulated loop reactor, a membrane reactor, such as a Hollow Fibre Membrane Bioreactor (HFM BR) or other vessel or other device suitable for gas-liquid contact. The reactor is preferably adapted to receive a gaseous substrate comprising CO or CO₂ or H₂ or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

Nutrient media or Nutrient medium is used to describe bacterial growth media. Generally, this term refers to a media containing nutrients and other components appropriate for the growth of a microbial culture. The term “nutrient” includes any substance that may be utilised in a metabolic pathway of a microorganism. Exemplary nutrients include potassium, B vitamins, trace metals and amino acids.

The term fermentation broth or broth is intended to encompass the mixture of components including nutrient media and a culture or one or more microorganisms. It should be noted that the term microorganism and the term bacteria are used interchangeably throughout the document.

The term “co-substrate” refers to a substance that, while not necessarily being the primary energy and material source for product synthesis, can be utilised for product synthesis when added to another substrate, such as the primary substrate.

The term “product” or “fermentation product” as used herein is intended to encompass substances produced by the microbial fermentation. Products can include alcohols, acids or other chemicals. Products can also include gases produced by the microbial fermentation process

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “desired composition” is used to refer to the desired level and types of components in a substance, such as, for example, of a gas stream. More particularly, a gas is considered to have a “desired composition” if it contains a particular component (e.g. CO and/or CO₂) and/or contains a particular component at a particular level and/or does not contain a particular component (e.g. a contaminant harmful to the micro-organisms) and/or does not contain a particular component at a particular level. More than one component may be considered when determining whether a gas stream has a desired composition.

The term “stream” is used to refer to a flow of material into, through and away from one or more stages of a process, for example, the material that is fed to a bioreactor and/or an optional CO₂ remover. The composition of the stream may vary as it passes through particular stages. For example, as a stream passes through the bioreactor, the CO content of the stream may decrease, while the CO₂ content may increase. Similarly, as the stream passes through the CO₂ remover stage, the CO₂ content will decrease.

Unless the context requires otherwise, the phrases “fermenting”, “fermentation process” or “fermentation reaction” and the like, as used herein, are intended to encompass both the growth phase and product biosynthesis phase of the process.

The terms “increasing the efficiency”, “increased efficiency” and the like, when used in relation to a fermentation process, include, but are not limited to, increasing one or more of: the rate of growth of micro-organisms in the fermentation, the volume or mass of desired product (such as alcohols) produced per volume or mass of substrate (such as carbon monoxide) consumed, the rate of production or level of production of the desired product, and the relative proportion of the desired product produced compared with other by-products of the fermentation, and further may reflect the value (which may be positive or negative) of any by-products generated during the process.

The term “BTEX”, “BTEX stream”, “BTEX components” and the like, as used herein, are intended to encompass industrial waste streams that contains at least a portion of one or more components selected from the group consisting of Benzene, Toluene, Ethyl Benzene and Xylene. As the skilled person would understand, the term is not limited to specific compositions of each of the components, and does not exclude other components from being present in the stream. The present invention relates to systems and methods for the production of one or more useful products by microbial fermentation of gaseous substrates comprising CO, or CO₂ and H₂ or mixtures thereof. The gaseous substrate can be derived from industrial processes. In certain aspects, the gaseous substrate is a waste gas stream derived from an industrial process.

The invention has particular applicability to supporting the production of ethanol, and/or 2,3-butanediol from gaseous substrates comprising naphthalene and BTEX constituents. Examples include gases produced during ferrous metal products manufacturing, biomass synthesis gas, coke oven gas, coal syngas, municipal solid waste syngas and COREX gas. In a particular embodiment of the invention, the waste gases are generated during a process for making steel. For example, those skilled in the art will appreciate the waste gases produced during various stages of the steel making process have high CO and/or CO₂ concentrations. In particular, the waste gas produced during the decarburisation of steel in various methods of steel manufacturing, such as in an oxygen converter (e.g. BOF or KOBM), has a high CO content and low O2 content making it a suitable substrate for anaerobic carboxydotrophic fermentation.

Waste gases produced during the carburisation of steel are optionally passed through water to remove particulate matter before passing to a waste stack or flue for directing the waste gas into the atmosphere. Typically, the gases are driven into the waste stack with one or more fans. In particular embodiments of the invention, at least a portion of the waste gas produced during the decarburisation of steel is diverted to a fermentation system by suitable conduit means. By way of example, a conduit means, i.e. piping or other transfer means can be connected to the waste gas stack from a steel mill to divert at least a portion of the waste gas to a fermentation system. Again, one or more fans can be used to divert at least a portion of the waste gas into the fermentation system. The conduit means is adapted to provide at least a portion of the waste gas produced during the decarburisation of steel to a fermentation system. The control of and means for feeding gases to a bioreactor will be readily apparent to those of ordinary skill in the art to which the invention relates.

While steel mills can be adapted to substantially continuously produce steel and subsequently waste gases, particular aspects of the process may be intermittent. Typically the decarburisation of steel is a batch process lasting several minutes to several hours. As such, the conduit means may be adapted to divert at least a portion of the waste gas, such as the gas produced during the decarburisation of steel, to the fermentation system if it is determined the waste gas has a desirable composition.

Typically the gases will contain additional material resulting from the industrial process. Certain constituents of the gas stream, may be used, at least in part, as a feedstock for the fermentation reaction.

### Pre-Treatment of the CO Containing Substrate

Industrial gas streams contain a wide variety of contaminant constituents including but not limited to ethane, acetylene, tar, ash, char particles, benzene, toluene, ethyl benzene, xylene, naphthalene and gases such as sulphur and nitrogen. By way of example, Table 1 shows all air emissions (Point source+Fugitive¹) in Kilograms from the BlueScope Steel Port Kembla Steelworks—Port Kembla, NSW, Australia as reported in the National Pollution Inventory (NPI)(http://www.npi.gov.au). This details the typical pollution causing components of off gases from the BlueScope Steel Port Kembla Steelworks—Port Kembla, NSW, Australia.

TABLE 1
Contaminants Present in an Exhaust Gas from
the BlueScope Steel Port Kembla Steelworks.

| | Air Total |
|---|---|
| Substance | (kg/yr) |
| Oxides of Nitrogen | 7927779 |
| Sulfur dioxide | 7498915 |
| Particulate Matter 10.0 um | 1722175 |
| Ammonia (total) | 735551 |
| Sulfuric acid | 259163 |
| Total Volatile Organic Compounds | 240305 |
| Hydrochloric acid | 190953 |
| Benzene | 130905 |
| Particulate Matter 2.5 um | 110063 |
| Hydrogen sulphide | 81748 |
| Toluene (methylbenzene) | 20220 |
| Cyanide (inorganic) compounds | 19483 |
| Fluoride compounds | 16780 |
| Methanol | 12131 |
| Methyl isobutyl ketone | 10775 |
| Zinc and compounds | 8228 |
| Manganese & compounds | 4001 |
| Chlorine & compounds | 3221 |
| Xylenes (individual or mixed isomers) | 2583 |
| Lead & compounds | 2391 |
| n-Hexane | 1142 |
| Styrene (ethenylbenzene) | 900 |
| ‘Copper & compounds | 575 |
| Cadmium & compounds | 425 |
| Nickel & compounds | 323 |
| Boron & compounds | 247 |
| Polycyclic aromatic hydrocarbons (B[a]Peq) | 192 |
| Chromium (III) compounds | 176 |
| Mercury & compounds | 168 |
| Ethanol | 123 |
| 1,3-Butadiene (vinyl ethylene) | 120 |
| Phenol | 115 |
| Selenium & compounds | 112 |
| Chromium (VI) compounds | 69 |
| Biphenyl (1,1-biphenyl) | 60 |
| Arsenic & compounds | 47 |
| Formaldehyde (methyl aldehyde) | 47 |
| Acetone | 26 |
| Antimony & compounds | 18 |
| Ethyl benzene | 18 |
| Carbon disulfide | 13 |
| Cobalt & compounds | 8 |
| Beryllium & compounds | 2 |
| Nitric acid | 1 |
| Polychlorinated dioxins and furans (TEQ) | 1.35E−04 |

Whilst the applicant's fermentation process has been demonstrated to operate in the presence of such constituents, it has been found that by limiting one or more of the constituent products such that they are present in the liquid fermentation broth within a predetermined range, the performance and stability of the process can be optimised. It has been found that by limiting the BTEX contaminants (benzene, ethylbenzene, xylene and toluene) to within a predetermined range, the stability of applicant's fermentation process can be optimised.

Pressure Swing Adsorption (PSA) can be used to remove BTEX contaminants from gaseous streams. Pressure swing adsorption (PSA) is an adiabatic process which may be used for the purification of gases to remove accompanying impurities by adsorption through suitable adsorbents in fixed beds contained in pressure vessels under high pressure. Regeneration of adsorbents is accomplished by counter current depressurization and by purging at low pressure with previously recovered near product quality gas. To obtain a continuous flow of product, preferably at least two adsorbers are provided, such that at least one adsorber is receiving a gas stream (such as a waste/exhaust/biogas gas stream) and actually produces a product of desired purity. Simultaneously, the subsequent steps of depressurization, purging and repressurization back to the adsorption pressure are executed by the other adsorber(s). Common adsorbents may readily be selected by one of skill in the art dependent on the type of impurity to be adsorbed and removed. Suitable adsorbents include zeolitic molecular sieves, activated carbon, silica gel or activated alumina. Combinations of adsorbent beds may be used on top of one another, thereby dividing the adsorber contents into a number of distinct zones. Pressure swing adsorption involves a pendulating swing in parameters such as pressure, temperature, flow and composition of gaseous and adsorbed phase.

Purification or separation of gases using PSA normally takes place at near ambient feed gas temperatures, whereby the components to be removed are selectively adsorbed. Adsorption should ideally be sufficiently reversible to enable regeneration of adsorbents at similar ambient temperature. PSA may be used for treatment and/or purification of most common gases including CO, CO₂ and H₂. Examples of Pressure Swing Adsorption techniques are described in detail in Ruthven, Douglas M. et al., 1993 Pressure Swing Adsorption, John Wiley and Sons.

In use the applicants found that using the PSA for separation of components of a gas stream comprising BTEX and naphthalene, the naphthalene component of the gas stream caused fouling of the silica gel in the PSA, thereby preventing the PSA from adsorbing the BTEX contaminants. When naphthalene adsorbs to the silica bed, a hotter regeneration temperature is then required to volatilize the naphthalene. This also requires that the cycle time of the PSA be increased such that the cycle provides sufficient time to raise the PSA to the required temperature.

Surprisingly it has been found that the addition of naphthalene to the reactor does not have a negative effect on the fermentation. However as naphthalene has a higher adsorption affinity to the PSA material, the presence of naphthalene in the gas stream results in an increased level of BTEX in the treated gas stream. As such, an additional step for removing naphthalene prior to the gas entering the PSA/TSA module has been added.

### Activated Carbon Bed.

In order to reduce the amount of naphthalene being passed to the PSA/TSA unit, an activated carbon bed was provided prior to the adsorption stage. The activated carbon beds can be regenerated with hot nitrogen. In use the beds are cycled such that one bed is active, whilst a second bed is regenerated.

Typically, nitrogen gas is used to regenerate the carbon beds. The use of nitrogen to regenerate the beds would not be feasible at large scale. Surprisingly, it has been found that it CO₂ captured in the PSA/TSA stage can be recycled to the carbon bed and used as the regeneration gas.

### Combined PSA/TSA Unit.

In use, the large amount of CO₂ adsorbed during the PSA cycle and the heat requirements of the adsorption/desorption, resulted in an inferior adsorption capacity of all adsorbable components. This can result in high outlet gas temperatures during adsorption and low gas outlet temperatures during desorption (regeneration). Higher temperature during adsorption decreases adsorption capacity for all adsorbable components including BTEX and naphthalene, while low desorption temperature allows more of the adsorbed species to remain adsorbed, especially BTEX and naphthalene.

To overcome this issue, applicants modified the PSA to a combine a PSA and Thermal Swing Adsorption (TSA) unit. In use, the cycle time, regeneration temperature and regenerant gas flow are set by the design rules for the TSA. Additionally it was found that as CO₂ saturates the PSA quickly, operating the unit with longer running times would provide a more constant gas composition exiting the adsorption unit. As the PSA adsorbent rapidly becomes saturated with CO₂, once the adsorbent becomes saturated, the gas composition for the remainder of the cycle is constant. An increased cycle time will ensure that a gas supply with a substantially constant composition is fed to the bioreactor for a longer period of time.

Operating with an imposed temperature swing increased the dynamic capacity, which is the difference in adsorbed amount during adsorption and desorption, for BTEX and naphthalene. In addition, an optimally designed TSA cycle could also remove all of the CO₂ from the feed gas, providing an additional benefit of reducing the amount of “diluent” CO₂ flowed to the reactor. Reducing the CO₂ in the gas stream provided to the fermentation process in effect enhances the CO and hydrogen composition of the gas stream as it increases the partial pressure of CO & H₂. This increase in CO and H₂ partial pressure enables more efficient production of desired fermentation products for a fixed amount of raw feed gas. Additionally this also increases the production capacity as the reactor ultimately has a gas volumetric throughput limitation.

A typical TSA design is meant to remove adsorbable materials that are desorbed at higher temp into an inert regenerant gas stream then condensed into liquid at lower temp in an external cooler, and removed from the regenerant gas in a separator, with the regenerant gas recycled via a blower and heated to remove more adsorbed components. Very large amounts of gas are required to heat and cool the adsorbent bed. In use approximately 1 kg of gas is required for each kg of adsorbent as the heat capacities of gas and adsorbent are nearly equal. Typically the regenerant gas loop is a substantially closed loop comprised of a cheap, non-adsorbable gas, such as nitrogen gas.

In order to overcome the problems described above, the combined PSA/TSA unit is designed to be able to use CO₂ as the regenerant gas so that it would be purged out of the TSA regenerant loop. This allows the naphthalene to exit the regenerant loop as a vapor. The BTEX partially condenses in the cooler, and in some embodiments at least a portion of the BTEX would exit the cooler as a vapor with the exiting CO₂. The contaminant stream comprising of CO₂, BTEX and naphthalene can be flared.

The substrate stream exiting the combined PSA/TSA unit is a BTEX, CO₂ and naphthalene depleted stream. In certain aspects the PSA/TSA unit operates to provide a gas stream containing BTEX in a composition that allows the concentration of BTEX in the fermentation broth to be maintained below a predetermined level. In certain embodiments the BTEX concentration in the fermentation broth is less than 100 ppm, or less than 80 ppm, or less than 60 ppm, or less than 40 ppm, or less than 20 ppm.

It is considered that at least a portion of the one or more BTEX contaminants separated from the gas stream can be recovered. For example, whilst benzene has been shown by the present inventors, to have a negative effect on the fermentation process, it is in its own right a valuable chemical commodity. By providing a means for the separation and recovery of one or more BTEX components, the economics of the fermentation process can be improved. The separation and recovery of Benzene can be carried out using known means. For example, condensation can be used to separate and recover one or more liquid components from a gaseous stream. The liquid component stream can then be treated by fractionation to recover at least a portion of one or more components selected from the group comprising benzene, toluene and xylene.

### Impact Levels of BTEX and Naphthalene Contaminants on the Fermentation.

It has been shown that the applicants fermentation process is capable of producing liquid products including ethanol, 2,3-butanediol and acetate by fermenting raw industrial gas stream containing large amounts of contaminant constituents.

The applicants have identified that by maintaining the level of BTEX constituents in the fermentation broth below a determined level, the performance and stability of the fermentation can be improved.

BTEX typically have the ability to quickly accumulate in liquid, and will accumulate to saturation. The accumulation levels of the BTEX constituents vary depending on whether they are added together or added to solution individually. This indicates that the BTEX constituents compete for solubility in solution. Furthermore, the applicant has found that the effect of BTEX on the fermentation is dependent on the biomass and ethanol concentrations in the broth. Higher ethanol concentrations in the broth appear to enable greater solubility of BTEX constituents. Therefore, in fermentations with low ethanol concentration, the amount of BTEX provided in the gas stream can be greater than in fermentations with higher ethanol concentrations. In view of this, applicant's process requires maintaining preferred liquid levels of BTEX. As such, it would be understood that the composition of BTEX in the treated gaseous stream can vary depending on the ethanol concentration in the fermentation. It would be further understood that the tolerance of the fermentation to BTEX in the gas stream will be greater during the early stage of the fermentation (i.e. start-up) when the ethanol concentration is lower. In certain aspects, the BTEX composition in the gas stream can be higher at the beginning of the fermentation. In certain aspects of the invention it may be desirable to monitor the ethanol concentration in the broth, and alter the composition of BTEX in the gas stream in response to increases or decreases in ethanol concentration.

It has been identified that excess levels of BTEX in the fermentation solution cause an increase in pH and can be detrimental to the fermentation. However, applicants surprisingly found that only toluene appears to have a clear negative effect on carbon monoxide utilisation of the microorganism. The applicants have also found that the compound effect of BTEX on the microorganism is reversible.

The fermentation may be carried out in any suitable bioreactor, such as a continuous stirred tank reactor (CSTR), an immobilised cell reactor, a gas-lift reactor, a bubble column reactor (BCR), a membrane reactor, such as a Hollow Fibre Membrane Bioreactor (HFM BR) or a trickle bed reactor (TBR). Also, in some embodiments of the invention, the bioreactor may comprise a first, growth reactor in which the micro-organisms are cultured, and a second, fermentation reactor, to which fermentation broth from the growth reactor may be fed and in which most of the fermentation product (e.g. ethanol and acetate) may be produced. The bioreactor of the present invention is adapted to receive a CO and/or H₂ containing substrate.

A substrate comprising carbon monoxide, preferably a gaseous substrate comprising carbon monoxide, is used in the fermentation reaction to produce ethanol in the methods of the invention. The gaseous substrate may be a waste gas obtained as a by-product of an industrial process, or from some other source such as from combustion engine (for example automobile) exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. Depending on the composition of the gaseous substrate comprising carbon monoxide, it may also be desirable to treat it to remove any undesired impurities, such as dust particles before introducing it to the fermentation. For example, the gaseous substrate may be filtered or scrubbed using known methods.

The gaseous substrate may be sourced from the gasification of organic matter such as methane, ethane, propane, coal, natural gas, crude oil, low value residues from oil refinery (including petroleum coke or petcoke), solid municipal waste, or biomass. Biomass includes by-products obtained during the extraction and processing of foodstuffs, such as sugar from sugarcane, or starch from maize or grains, or non-food biomass waste generated by the forestry industry. Any of these carbonaceous materials may be gasified, i.e., partially combusted with oxygen, to produce synthesis gas (syngas). Syngas typically comprises mainly CO, H₂, and/or CO₂ and may additionally contain amounts of methane, ethylene, ethane, or other gasses. The operating conditions of the gasifier can be adjusted to provide a substrate stream with a desirable composition for fermentation or blending with one or more other streams to provide an optimised or desirable composition for increased alcohol productivity and/or overall carbon capture in a fermentation process.

The gaseous substrate may be sourced from a pressure swing adsorption (PSA) system. For example, a PSA tail gas may contain ˜10-12% of the H₂ entering the PSA from a methane steam reformer, in addition to CO and CO₂ from the water-gas shift reactors in the methane steam reformer. CO in a gas exiting a primary methane reformer (at about 3 H₂/CO) may be reacted with water to form H₂ and CO₂ using water-gas shift reactors (high temperature and low temperature). The reaction conditions may be tailored to control the amount of CO present in the PSA tail gas relative to the amount of CO₂ present in the PSA tail gas. It may also be desirable to allow some of the H₂ to remain in the PSA tail gas, or to add H₂ back to the PSA tail gas, to achieve a desirable H₂/CO/CO₂ ratio

The CO-containing substrate will typically contain a major proportion of CO, such as at least about 15% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

The gaseous substrate may also contain some CO₂ for example, such as about 1% to about 80% by volume, or 1% to about 30% by volume. In one embodiment it contains about 5% to about 10% by volume. In another embodiment the gaseous substrate contains approximately 20% CO₂ by volume.

Typically, the carbon monoxide will be added to the fermentation reaction in a gaseous state. However, the invention should not be considered to be limited to addition of the substrate in this state. For example, the carbon monoxide could be provided in a liquid. For example, a liquid may be saturated with a carbon monoxide containing gas and then that liquid added to a bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used.

In one embodiment of the invention, a combination of two or more different substrates may be used in the fermentation reaction.

In addition, it is often desirable to increase the CO concentration of a substrate stream (or CO partial pressure in a gaseous substrate) and thus increase the efficiency of fermentation reactions where CO is a substrate. Increasing CO partial pressure in a gaseous substrate increases CO mass transfer into a fermentation media. The composition of gas streams used to feed a fermentation reaction can have a significant impact on the efficiency and/or costs of that reaction. For example, O₂ may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

It may be desirable to blend a reformed substrate stream comprising CO and H₂ with one or more further streams in order to improve efficiency, alcohol production and/or overall carbon capture of the fermentation reaction. Without wishing to be bound by theory, in some embodiments of the present invention, carboxydotrophic bacteria convert CO to ethanol according to the following:

6CO+3H₂O→C₂H₅OH+4CO₂

However, in the presence of H₂, the overall conversion can be as follows:

6CO+12H₂→3C₂H₅OH+3H₂O

Accordingly, streams with high CO content can be blended with reformed substrate streams comprising CO and H₂ to increase the CO:H₂ ratio to optimise fermentation efficiency. By way of example, industrial waste streams, such as off-gas from a steel mill have a high CO content, but include minimal or no H₂. As such, it can be desirable to blend one or more streams comprising CO and H₂ with the waste stream comprising CO, prior to providing the blended substrate stream to the fermenter. The overall efficiency, alcohol productivity and/or overall carbon capture of the fermentation will be dependent on the stoichiometry of the CO and H₂ in the blended stream. However, in particular embodiments the blended stream may substantially comprise CO and H₂ in the following molar ratios: 20:1, 10:1, 5:1, 3:1, 2:1, 1:1 or 1:2.

In addition, it may be desirable to provide CO and H₂ in particular ratios at different stages of the fermentation. For example, substrate streams with a relatively high H₂ content (such as 1:2 CO:H₂) may be provided to the fermentation stage during start up and/or phases of rapid microbial growth. However, when the growth phase slows, such that the culture is maintained at a substantially steady microbial density, the CO content may be increased (such as at least 1:1 or 2:1 or higher, wherein the H₂ concentration may be greater or equal to zero).

Blending of streams may also have further advantages, particularly in instances where a waste stream comprising CO is intermittent in nature. For example, an intermittent waste stream comprising CO may be blended with a substantially continuous reformed substrate stream comprising CO and H₂ and provided to the fermenter. In particular embodiments of the invention, the composition and flow rate of the substantially continuous blended stream may be varied in accordance with the intermittent stream in order to maintain provision of a substrate stream of substantially continuous composition and flow rate to the fermenter.

### Media

It will be appreciated that for growth of the one or more microorganisms and substrate to ethanol and/or acetate fermentation to occur, in addition to the substrate, a suitable nutrient medium will need to be fed to the bioreactor. A nutrient medium will contain components, such as vitamins and minerals, sufficient to permit growth of the micro-organism used. By way of example only, anaerobic media suitable for the growth of Clostridium autoethanogenum are known in the art, as described for example by Abrini et al (Clostridium autoethanogenum, sp. Nov., An Anaerobic Bacterium That Produces Ethanol From Carbon Monoxide; Arch. Microbiol., 161: 345-351 (1994)). The “Examples” section herein after provides further examples of suitable media.

### Fermentation

Processes for the production of ethanol and other alcohols from gaseous substrates are known. Exemplary processes include those described for example in WO2007/117157, WO2008/115080, WO2009/022925, WO2009/064200, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

### Fermentation Conditions

The fermentation should desirably be carried out under appropriate conditions for the substrate to ethanol and/or acetate fermentation to occur. Reaction conditions that should be considered include temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in general, it is preferred that the fermentation be performed at a pressure higher than ambient pressure. Operating at increased pressures allows a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

Also, since a given CO-to-product conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-product fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

Examples of fermentation conditions suitable for anaerobic fermentation of a substrate comprising CO are detailed in WO2007/117157, WO2008/115080, WO2009/022925 and WO2009/064200. It is recognised the fermentation conditions reported therein can be readily modified in accordance with the methods of the instant invention.

In various embodiments, the fermentation is carried out using a culture of one or more strains of carboxydotrophic bacteria. In various embodiments, the carboxydotrophic bacterium is selected from Moorella, Clostridium, Ruminococcus, Acetobacterium, Eubacterium, Butyribacterium, Oxobacter, Methanosarcina, Methanosarcina, and Desulfotomaculum. A number of anaerobic bacteria are known to be capable of carrying out the fermentation of CO to alcohols, including n-butanol and ethanol, and acetic acid, and are suitable for use in the process of the present invention. Examples of such bacteria that are suitable for use in the invention include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091), Clostridium ragsdalei (WO/2008/028055) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Moorella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. pp 41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is a Clostridium autoethanogenum having the identifying characteristics of the strain deposited at the German Resource Centre for Biological Material (DSMZ) under the identifying deposit number 19630. In another embodiment, the Clostridium autoethanogenum is a Clostridium autoethanogenum having the identifying characteristics of DSMZ deposit number DSMZ 10061. These strains have a particular tolerance to changes in substrate composition, particularly of H₂ and CO and as such are particularly well suited for use in combination with a steam reforming process.

One exemplary micro-organism suitable for use in the production of acetate from a substrate comprising CO₂ and H₂ in accordance with one aspect of the present invention is Acetobacterium woodii.

Culturing of the bacteria used in the methods of the invention may be conducted using any number of processes known in the art for culturing and fermenting substrates using anaerobic bacteria. By way of example, those processes generally described in the following articles using gaseous substrates for fermentation may be utilised: (i) K. T. Klasson, et al. (1991). Bioreactors for synthesis gas fermentations resources. Conservation and Recycling, 5; 145-165; (ii) K. T. Klasson, et al. (1991). Bioreactor design for synthesis gas fermentations. Fuel. 70. 605-614; (iii) K. T. Klasson, et al. (1992). Bioconversion of synthesis gas into liquid or gaseous fuels. Enzyme and Microbial Technology. 14; 602-608; (iv) J. L. Vega, et al. (1989). Study of Gaseous Substrate Fermentation: Carbon Monoxide Conversion to Acetate. 2. Continuous Culture. Biotech. Bioeng. 34. 6. 785-793; (v) J. L. Vega, et al. (1989). Study of gaseous substrate fermentations: Carbon monoxide conversion to acetate. 1. Batch culture. Biotechnology and Bioengineering. 34. 6. 774-784; (vi) J. L. Vega, et al. (1990). Design of Bioreactors for Coal Synthesis Gas Fermentations. Resources, Conservation and Recycling. 3. 149-160; all of which are incorporated herein by reference.

The pH of the contents of the reactor may be adjusted as required. The appropriate pH will depend on the conditions required for a particular fermentation reaction, taking into account the liquid nutrition medium and the bacteria used. In a preferred embodiment involving the fermentation of a gaseous substrate containing H₂, CO₂, and CO by Clostridium autoethanogenum, the pH may be adjusted to approximately 4.5 to 6.5, most preferably to approximately 5 to 5.5. Further examples include a pH 5.5 to 6.5 using Moorella thermoacetica for the production of acetic acid, a pH 4.5 to 6.5 using Clostridium acetobutylicum for the production of butanol, and a pH 7 using Carboxydothermus hygrogenaformans for the production of hydrogen. Means for adjusting and maintaining the pH of a reactor are well known in the art. Such means may include the use of aqueous bases, such as NaOH or NH₄OH, and aqueous acids, such as H₂SO₄.

Preferably, the reactor is configured to provide enough mass transfer to allow the bacteria to access the gaseous substrate, particularly the H₂ in the gaseous substrate. Long gas residence times generate high gas uptake by the bacteria. In particular embodiments, the reactor is a circulated loop reactor comprising a riser segment and a downcomer segment through which the gaseous substrate and liquid nutrient media are circulated. The reactor may additionally include a wide range of suitable gas/liquid contact modules that can provide effective mass transfer. A contact module may provide a unique geometrical environment allowing gas and liquid to mix thoroughly along a set flow path, causing the entrained gas to dissolve in the liquid more uniformly. By way of example, this contact module may include, but is not limited to, a matrix of structured corrugated metal packing, random packing, sieve plates, and/or static mixers.

The mass transfer rate of the gaseous substrate to the bacterial culture may be controlled, so that the bacterial culture is supplied with gaseous substrate at or near an optimum supply rate. The mass transfer rate may be controlled by controlling the partial pressure of the gaseous substrate and/or by controlling the liquid flow rate or gas holdup. The rate of introduction of the gaseous substrate may be monitored to ensure that the concentration of H₂, CO₂, and/or CO in the liquid phase does not become limiting. In particular embodiments, the mass transfer is controlled by controlling the partial pressure of the gaseous substrate entering the reactor.

It may be preferable to perform the fermentation at elevated pressure, i.e., at a pressure higher than ambient pressure. Operating at increased pressures allows a significant increase in the rate of H₂, CO₂, and/or CO transfer from the gas phase to the liquid phase where it can be taken up by the bacteria as a carbon source for the production of products, such as ethanol. The retention time (the liquid volume in the bioreactor divided by the input gas flow rate) may be reduced when the reactor is maintained at elevated pressure rather than atmospheric pressure. Also, because a given CO/CO₂/H₂-to-ethanol conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure. For example, reactors operated at 10 atmospheres of pressure need only be one tenth the volume of those operated at 1 atmosphere of pressure. The benefits of conducting a gas-to-ethanol fermentation at elevated pressures have also been described elsewhere. For example, WO 2002/08438 describes gas-to-ethanol fermentations performed under pressures of 30 psig and 75 psig, giving ethanol productivities of 150 g/L/day and 369 g/L/day respectively. However, example fermentations performed using similar media and input gas compositions at atmospheric pressure were found to produce between 10 and 20 times less ethanol per litre per day.

### Fermentation Products

Methods of the invention can be used to produce any of a variety of products. Products may include alcohols, acids, or other chemicals, such products may also include gases produced by the fermentation processes. In particular, the culture may produce on or more of ethanol, acetic acid (acetate), 2,3-butanediol, butanol, isopropanol, isoprene, lactate, succinate, methyl ethyl ketone (MEK), propanediol, 2-propanol, acetoin, isobutanol, citramalate, butadiene, poly lactic acid, isobutylene, 3-hydroxy propionate (3HP), acetone, and fatty acids. In a preferred embodiment, the culture produces one or more of ethanol, acetate, and 2,3-butanediol. A skilled person would understand that the invention is not limited to the alcohols and products mentioned, any appropriate alcohol and or acid can be used to produce a product.

These and other products may be of value for a host of other processes such as the production of plastics, pharmaceuticals and agrochemicals. In one embodiment, the fermentation product is used to produce gasoline range hydrocarbons (about 8 carbon), diesel hydrocarbons (about 12 carbon) or jet fuel hydrocarbons (about 12 carbon).

The methods of the invention can also be applied to aerobic fermentations, to anaerobic or aerobic fermentations of other products, including but not limited to isopropanol. The methods of the invention can also be applied to aerobic fermentations, and to anaerobic or aerobic fermentations of other products, including but not limited to isopropanol.

The invention also provides that at least a portion of a hydrocarbon product produced by the fermentation is reused in the steam reforming process. This may be performed because hydrocarbons other than CH₄ are able to react with steam over a catalyst to produce H₂ and CO. In a particular embodiment, ethanol is recycled to be used as a feedstock for the steam reforming process. In a further embodiment, the hydrocarbon feedstock and/or product is passed through a prereformer prior to being used in the steam reforming process. Passing through a prereformer partially completes the steam reforming step of the steam reforming process which can increase the efficiency of hydrogen production and reduce the required capacity of the steam reforming furnace.

The methods of the invention can also be applied to aerobic fermentations, and to anaerobic or aerobic fermentations of other products, including but not limited to isopropanol.

More particularly, the invention may be applicable to fermentation to ethanol and/or acetate. These products may then be reacted to together to produce chemical products including esters. In one embodiment of the invention the ethanol and acetate produced by fermentation are reacted together to produce Ethyl Acetate. Ethyl acetate may be of value for a host of other processes such as the production of solvents including surface coating and thinners as well as in the manufacture of pharmaceuticals and flavours and essences.

### Product Recovery

The products of the fermentation reaction can be recovered using known methods. Exemplary methods include those described in WO07/117157, WO08/115080, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non-volatile and is recovered for re-use in the fermentation.

Acetate, which may be produced as a by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used. For example, U.S. Pat. Nos. 6,368,819 and 6,753,170 describe a solvent and co-solvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

The reactor may be integrated with a cell recycle system that provides a means for separating bacteria from the permeate so that the bacteria may be returned to the reactor for further fermentation. A cell recycle module may continuously draws broth permeate, while retaining cells. The cell recycle system may include, but is not limited to, cell recycle membranes and disc-stack centrifugal separators.

Biomass recovered from the bioreactor may undergo anaerobic digestion in a digestion. to produce a biomass product, preferably methane. This biomass product may be used as a feedstock for the steam reforming process or used to produce supplemental heat to drive one or more of the reactions defined herein.

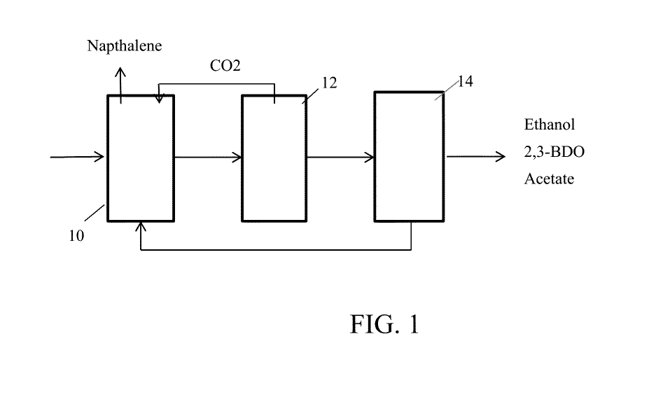

FIG. 1 shows one embodiment of the system and method of the present Invention. An industrial waste gas comprising CO, naphthalene and at least one contaminant selected from the group consisting of benzene, toluene, xylene and ethyl benzene, is passed to an activated carbon bed unit. The activated carbon bed unit (10) comprises two or more carbon beds which remove at least a portion of the naphthalene contained from the gas stream. The naphthalene depleted stream is then passed to a Pressure swing adsorption unit where at least a portion of the BTEX constituents is removed from the gaseous stream to provide a reactor stream. The reactor stream exits the PSA unit 14 via a PSA exit conduit 15. In accordance with certain aspects of the invention CO₂ and naphthalene are also removed from the stream in the PSA unit 12. The CO₂ exiting the PSA unit 12 can be passed back to the activated carbon bed 10 to act as the regenerating gas in the carbon bed unit. The reactor stream is substantially depleted of naphthalene and BTEX constituents. In certain embodiments of the invention the reactor stream is substantially free of naphthalene and/or BTEX. The reactor stream is passed to a bioreactor 14 comprising a culture of one or more microorganism. The reactor stream is fermented to produce one or more alcohols and or acids. An exit stream comprising unreacted gaseous components of the fermentation exits the bioreactor via an exit conduit 16. In certain aspects of the invention, the exit stream comprises CO₂. In one embodiment the CO₂ exiting the reactor is passed to the activated carbon bed 10 for use as a regeneration gas.

### EXAMPLES

TABLE 1
Media composition

| | Final concentration |
|---|---|
| Component | mM/L |
| KCl | 25 |
| CaCl₂•2H₂O | 2 |
| MgCl₂•6H₂O | 2 |
| NaCl | 2 |
| H₃PO₄ | 5 |
| Metal solution [Table 2] | 20 mL |
| Vitamin solution [Table 3] | 20 mL |

TABLE 2
Trace metals solution

| | Concentration mM/L |
|---|---|
| Metal | stock solution |
| FeCl₂•4H₂O | 20 |
| MnCl₂•4H₂O | 0.4 |
| CoCl₂•6H₂O | 1.0 |
| ZnCl₂ | 1.0 |
| H₃BO₃ | 1.0 |
| Na₂MoO₄•2H₂O | 0.4 |
| NiCl₂•6H₂O | 0.4 |
| Na₂SeO₃ | 0.4 |
| Na₂WO₄•2H₂O | 0.4 |

TABLE 3
Vitamin solution

| | Concentration mg/L |
|---|---|
| Vitamin | stock solution (100x) |
| Thiamine hydrochloride | 50 |
| (Vitamin B1) | |
| Riboavin (Vitamin B2) | 50 |
| Nicotinic acid (Niacin or | 50 |
| Vitamin B3) | |
| Pantothenic acid (Vitamin | 50 |
| B5) | |
| Pyridoxine hydrochloride | 10 |
| (Vitamin B6) | |
| Biotin (Vitamin B7) | 20 |
| Folic acid (Vitamin B9) | 20 |
| 4-Aminobenzoic acid (PABA | 50 |
| or Vitamin B10) | |
| Cyanocobalamin (Vitamin | 50 |
| B12) | |
| Lipoic acid (Thioctic acid) | 50 |

### Example 1

### Determination of Effect of BTEX on Fermentation

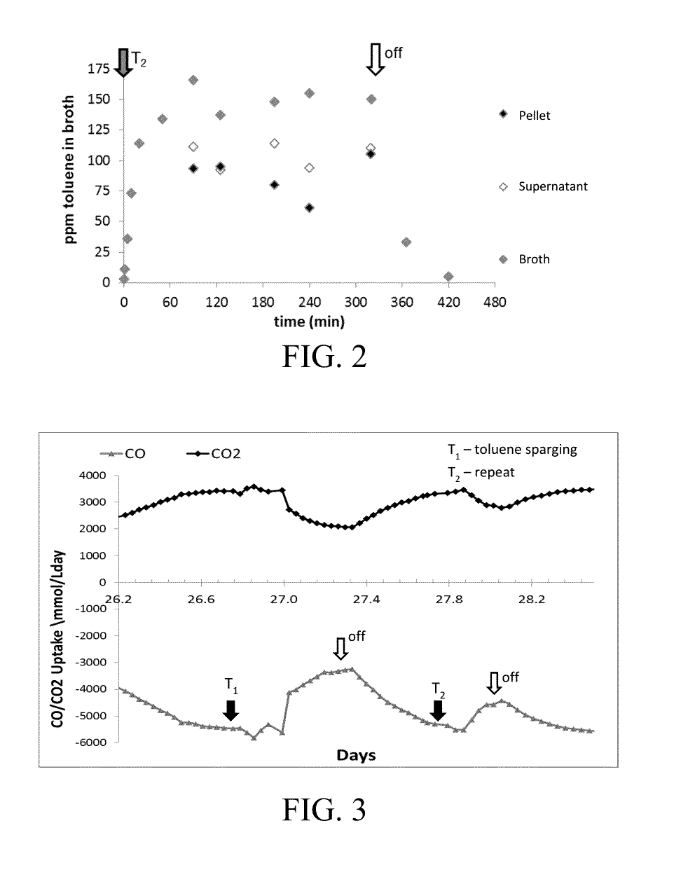

Media was prepared according to the composition described in Tables 1-3 to a volume of 1.5 L and 1.5 ml of resazurin added. The solution was heated and agitated whilst degassed with N₂. A Na₂S drip was started at a rate of 0.5 ml/hr and temperature of the bioreactor set to 37° C. The pH was adjusted to 5.0 with NH₄OH and chromium was added to adjust the ORP to −200 mV. The bioreactor was then supplied with RMG (43% CO, 20% CO₂, 2.5% H₂ and 33% N₂) at a flow rate of 50 ml/min. The solution was inoculated with 150 ml of an actively growing Clostridium autoethanogenum culture. The fermentation was operated continuously for a period of 41 days at dilution rate 1.5. At day 26.8, Toluene was added to the gas phase. To achieve this, the inflow gas was sparged through a toluene solution, which allowed toluene to accumulate in the reactor. Toluene was allowed to accumulate for 17 hours. The fermentation was allowed to recover and the experiment was repeated on day 27.8. FIG. 2 shows the accumulation profile of toluene over a period of 8 hours from day 27.8,

The reactor was allowed to recover. On day 35 Benzene was added to the gas phase. To achieve this, the inflow gas was sparged through a benzene solution, which allowed benzene to accumulate in the reactor. At an accumulation level of 40 ppm benzene or less, there was no observable effect of benzene on the fermentation. Benzene was allowed to accumulate to higher concentrations.

The effect of the addition of xylene and ethylbenzene were also performed using the same techniques. Based on vapour pressure the compounds would evaporate at a certain rate into gaseous phase.

### Results

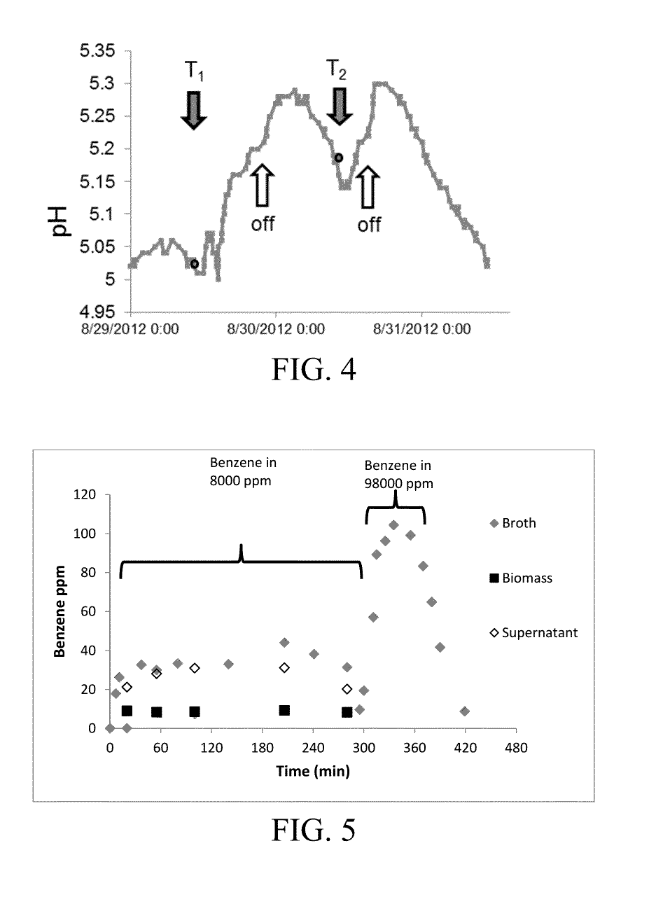

The addition of toluene to the reactor resulted in changes to the CO utilization and the pH in the bioreactor. FIG. 2 shows the levels of toluene (ppm) in the fermentation broth on day 30 of the fermentation. After 3.5 hours exposure, when the level of toluene in the fermentation broth measure at greater than 100 ppm, the CO uptake by the fermentation decreased, as seen in FIG. 3. This was accompanied by a rising pH trend, as seen in FIG. 4, which is indicative of cellular stress. Once toluene addition was stopped, toluene concentration decreases as the toluene is sparged out of the system. When the toluene level lowers below 100 ppm, CO uptake and pH returned to pre-toluene addition levels, and the fermentation recovers.

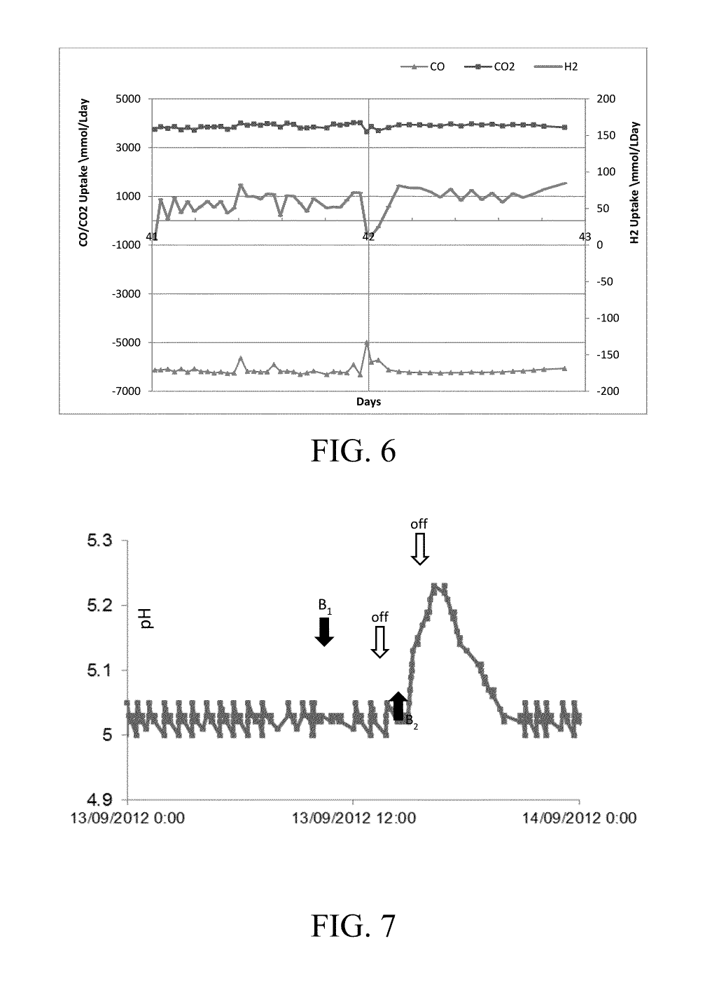

The presence of benzene in the broth began to effect the fermentation at levels of 50 ppm and above. Benzene accumulation of greater than 50 ppm resulted in an increase in pH and decrease in CO uptake. There is a clear correlation between increased benzene in the fermentation broth, and an increase in the pH of the fermentation (FIG. 7) indicating cellular stress. Increased levels of benzene in the fermentation broth also caused a decrease in CO uptake as seen in FIG. 6.

The same effect was also seen with addition of xylene and ethylbenzene.

The effect of the introduction of the various BTEX compounds on bacterial culture performance was determined by measurement of the following: culture density, metabolite concentration (ethanol, acetate, etc), oxidation-reduction potential, pH, carbon monoxide and hydrogen consumption. The compound accumulation in the gas and culture liquid phase was determined by GC-MS measurements.

Individual addition of BTEX compounds indicated the culture impact level for each compound. These results are summarised in Table 2.

TABLE 2
Liquid Impact Levels of BTEX components in the Fermentation Broth

| | mm Hg vapor | Liquid | Liquid impact |
|---|---|---|---|
| Solvent | pressure (25° C.) | tolerance level | level (ppm) |
| Benzene | 76 | <40 ppm | >40 ppm |
| Toluene | 22 | <25 ppm | >50 ppm |
| Xylenes | 5-6.5 | <10 ppm | >100 ppm |
| Ethyl Benzene | 7 | <10 ppm | >20 ppm |

An impact was observed either by a rising pH and/or effect on carbon monoxide and hydrogen uptake. Liquid tolerance levels were determined by lowering the addition until no effect was observed.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

What is claimed is:

1. A method for the production of one or more alcohols by microbial fermentation of a substrate comprising CO, the method comprising:
a) flowing an industrial waste gas stream comprising CO, naphthalene and at least one contaminant selected from the group consisting of benzene, toluene, xylene and ethyl benzene (BTEX) to an activated carbon bed unit, wherein at least a portion of the naphthalene is removed from the gas stream;
b) passing the naphthalene depleted gas stream of (a) to an adsorption unit, wherein at least a portion of the at least one BTEX contaminant is removed from the gas stream to provide a BTEX depleted reactor stream;
c) passing the BTEX depleted reactor stream to a bioreactor containing a fermentation broth comprising a culture of at least one microorganism; and
d) fermenting the BTEX depleted reactor stream to produce one or more products.

2. The method of claim 1 wherein the one or more products is selected from the group consisting of ethanol, 2,3-butanediol and acetate.

3. The method of claim 1 wherein the total BTEX contaminant concentration in the fermentation broth is less than 100 ppm.

4. The method of claim 3, wherein the total BTEX contaminant composition in the fermentation broth is less than 40 ppm.

5. The method of claim 3 wherein substantially no BTEX contaminants are present in the fermentation broth.

6. The method of claim 2, wherein the BTEX contaminant composition of the reactor stream is adjusted in response to the ethanol concentration present in the fermentation broth.

7. The method of claim 1 further comprising regenerating the activated carbon bed unit using a carbon dioxide gas stream.

8. The method of claim 1 wherein the naphthalene depleted stream further comprises CO₂ and the stream is flowed to an adsorption unit, wherein at least a portion of CO₂ in the naphthalene depleted stream is adsorbed.

9. The method of claim 8, wherein at least a portion of the adsorbed CO₂ is desorbed and passed to the activated carbon bed unit for use as a regeneration gas.

10. The method of claim 1 wherein CO₂ is produced as a by-product of the fermentation.

11. The method of claim 10 wherein at least a portion of the produced CO₂ is passed to the activated carbon bed unit for use as a regeneration gas.

12. The method of claim 1, wherein the at least one removed BTEX contaminant is recovered.

13. The method of claim 1 wherein the industrial waste gas is selected from the group consisting of; ferrous metal product manufacturing process waste gas, biomass synthesis gas, coke oven gas, coal syngas, municipal solid waste syngas and COREX gas.

14. The method of claim 13 wherein the industrial waste gas is a steel manufacturing process waste gas.

15. The method of claim 1 where the adsorption unit is a Pressure Swing Adsorption (PSA) unit.

16. The method of claim 1 where the adsorption unit is a combined Temperature Swing Adsorption (TSA) and a Pressure Swing Adsorption (PSA) unit.
